(19) United States
(12) Patent Application Publication
(10) Pub. No.: US 2002/0058243 A1
(43) Pub. Date: May 16, 2002

(54) Rapid, parallel identification of cell lines

(76) Inventors: Kurt Jarnigan, San Mateo, CA (US); Hua Zhou, Santa Clara, CA (US)

Correspondence Address: ROBINS & PASTERNAK LLP, 90 MIDDLEFIELD ROAD, SUITE 200, MENLO PARK, CA, 94025, US

(21) Appl. No.: 09/776,167

(22) Filed: Feb. 2, 2001

Related U.S. Application Data
(60) Provisional application No. 60/179,893, filed on Feb. 2, 2000.

(51) Int. Cl. C12Q 1/00; C12Q 1/68; C07H 21/04; C12N 5/00

(52) U.S. Cl. 435/4; 435/6; 435/325; 536/23.1

(57) ABSTRACT

Host cells that exhibit regulated expression of a test gene, are rapidly identifyed by providing a plurality of host cells, each host cell comprising a polynucleotide, the polynucleotide comprising in order a regulatable promoter; a test gene; an IRES sequence; and a surface marker coding sequence, where the surface marker comprises a secretion signal sequence, a detectable label protein, and a membrane anchor; wherein expression of said test gene also results in expression of said surface marker; inducing said promoter; and selecting a host cell that displays said surface marker on its surface.

### FIELD OF THE INVENTION

This invention is related generally to the fields of molecular biology and genomics. More particularly, the invention relates to nucleic acid vectors and methods for rapidly identifying transformed cells with regulated expression of a heterologous polynucleotide.

### BACKGROUND OF THE INVENTION

Identification of clonal eukaryotic cells lines that have regulated expression of target cDNAs or genes has been a time consuming and expensive process. During the course of cell biological and functional genomic investigations it is often desirable to create cell lines in which a cDNA is expressed in a regulated manner. For this purpose many types of regulons (combinations of transactivators and regulated promoters) have been invented. These include the tetracycline regulon (U. Baron, et al., Nuc Acids Res (1995) 23(17):3605-06), the ecdysone regulon (D. No, et al., Proc Natl Acad Sci USA (1996) 93(8):3346-51), regulons controlled by a transplanted E. coli Lac/La repressor system, the heat shock regulon, and the metalothionine regulon. These systems provide, with some effort, clonal cell lines in which the cDNA is regulated by application of an exogenous stimulator: tetracycline, ecdysone, isopropylthiogal-actopyranoside (ITGP), heat or heavy metals, respectively. The time and expense of creating these cells lines arises from the need to isolate and expand numerous single cell clones and analyze each clone for appropriate regulation of the query gene. If a way could be devised to use a rapid cell sorting technique such as flow cytometry, cell panning, colorimetric overlays or magnetic selection to identify candidate regulated clones, significant time and effort could be saved.

### SUMMARY OF THE INVENTION

We have now invented a construct and method for rapidly labeling host cells, and for determining which cells provide regulated expression of a test gene (and surface label).

One aspect of the invention is a polynucleotide, comprising a regulatable promoter; a test gene; an IRES sequence; a surface marker coding sequence, said surface marker comprising a secretion signal sequence, a detectable label protein, and a membrane anchor; wherein expression of said test gene also results in expression of said surface marker.

Another aspect of the invention is a host cell, comprising: a polynucleotide, said polynucleotide comprising a regulatable promoter, a test gene, an IRES sequence, a surface marker coding sequence, said surface marker comprising a secretion signal sequence, a detectable label protein, and a membrane anchor; wherein expression of said test gene also results in expression of said surface marker.

Another aspect of the invention is a method of identifying a host cell that exhibits regulated expression of a test gene, comprising: providing a plurality of host cells, each host cell comprising a polynucleotide, said polynucleotide comprising in order a regulatable promoter; a test gene; an IRES sequence; and a surface marker coding sequence, said surface marker comprising a secretion signal sequence, a detectable label protein, and a membrane anchor; wherein expression of said test gene also results in expression of said surface marker; inducing said promoter; and selecting a host cell that displays said surface marker on its surface.

Another aspect of the invention is a method of identifying a host cell that exhibits regulated expression of a test gene, comprising: providing a plurality of host cells, each host cell comprising a polynucleotide, said polynucleotide comprising in order a regulatable promoter; a test gene; an IRES sequence; and a surface marker coding sequence, said surface marker comprising a secretion signal sequence, a detectable label protein, and a membrane anchor; wherein expression of said test gene also results in expression of said surface marker; selecting a plurality of host cells that do not exhibit said surface marker in the absence of induction of said promoter; inducing said promoter; and selecting a host cell that exhibits expression of said surface marker.

### BRIEF DESCRIPTION OF THE FIGURES

FIG. 1 is a diagram of an embodiment of the invention (the pFastFind vector, SEQ ID NO:5).

FIG. 2 is a diagram of the pFastFind-delta vector (SEQ ID NO:6), a version having a reduced size surface epitope marker.

FIG. 3 is a diagram of the pFastFind-point vector (SEQ ID NO:7), a version with a point mutation that eliminates the alkaline phosphatase enzymatic activity from the surface epitope marker while maintaining the size and epitope characteristics.

FIG. 4 is a summary protocol for isolation of regulated expression clones using vectors of the invention.

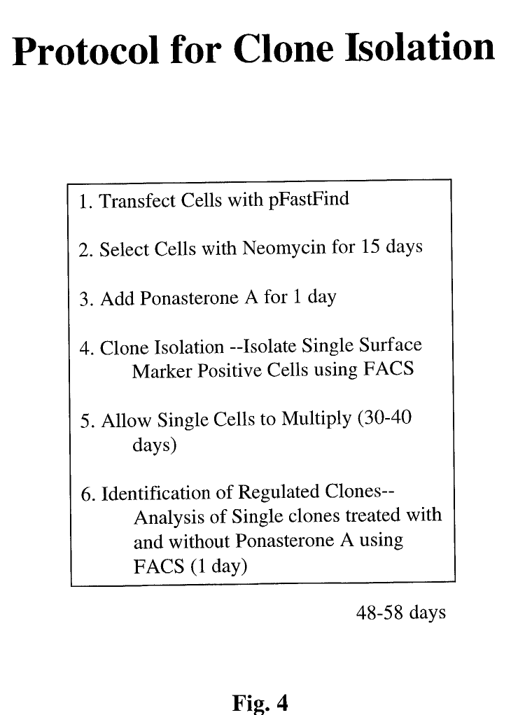

FIG. 5 presents data demonstrating that pFastFind results in the preparation of cell lines with regulated expression that persists for 36 hours after ponasterone-A treatment, and is induced by micromolar concentrations of ponasterone-A.

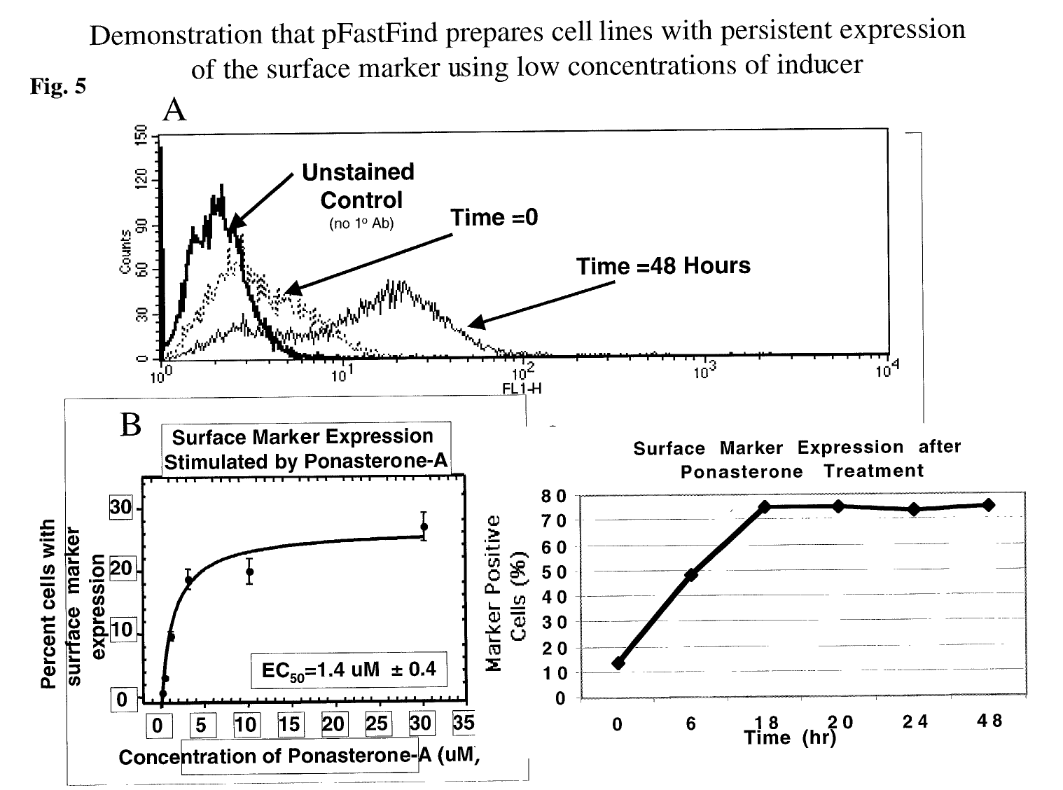

FIG. 6 presents data demonstrating that the surrogate marker expression correlates with the amount of mRNA encoding the query gene.

### DETAILED DESCRIPTION

Definitions:

The terms “test gene” and “query gene” refer to a polynucleotide to be examined, whether its function is known or unknown, regardless of whether it is synthetic or identical to a known sequence.

The term “IRES” refers to an internal ribosome binding site, or other sequence capable of serving as a translational initiation point when transcribed into mRNA.

The term “surface marker” refers to a protein which is associated with the surface of a host cell following translation. In general, the surface marker must be detectable either directly or through binding a labeled binding partner.

The term “regulatable promoter” refers to a polynucleotide sequence capable of controlling the transcription of an adjacent polynucleotide, and which can be controlled by altering or adjusting the host cell's environment. The environment can be adjusted by addition or subtraction of various factors or compounds, by altering the temperature, pressure, concentration of media components, radiation, and the like.

The term “FACS” refers to fluorescence-activated cell sorting, and includes any method for separating cells on the basis of a visible or fluorescent label. The label can be attached directly to the cell (for example, it can be expressed as a cell surface protein), or can be bound to the cell surface (for example, by allowing a labeled antibody to recognize and bind to a cell surface antigen).

General Method:

The vector of the invention and its method of use allows rapid isolation of candidate eukaryotic cell clones in which a query gene is regulated by exogenous application of an appropriate stimulus. The vector is arranged such that the query gene can be cloned immediately downstream of the regulated promoter by means of a multiple cloning site (MCS). Downstream of the multiple cloning site is placed an internal ribosome entry site (IRES); and downstream of the IRES is placed a cell membrane-localized protein for which an epitope recognized by a convenient antibody is available (a surrogate surface marker). Thus, since the surface epitope is co-cistronic with the query gene, both the query gene and the surface epitope will be elevated in response to the exogenous stimulator.

The use of a surrogate surface marker for the query gene allows isolation of clonal cell lines with stimulator-induced expression by means of flow cytometry, magnetic cell sorting, cell panning, cell enrichment by column chromatography, by use of colorimetric cell overlay methods, and other cell enrichment techniques. The use of a surrogate surface marker for the query gene circumvents any need for a specific antibody to the query gene's encoded protein, it circumvents any need for a biochemical assay for the query gene's product. The surrogate surface marker allows rapid reconfirmation of the regulation and expression of the query gene by use of the above mentioned techniques.

Suitable surrogate surface markers include, without limitation, placental alkaline phosphatase, β-lactamase, β2-microglobulin, and the like. If desired, one can select or construct any distinct surface protein, and prepare antibodies capable of recognizing the protein by conventional methods. The polynucleotide encoding the surface marker preferably further includes a secretion signal sequence (or other sequence that provides for export of the protein to the outer surface of the cell), and a transmembrane anchor (or other sequence that insures that the protein will remain associated with the cell surface). The surface marker is preferably relatively non-toxic to the cell. The surface marker can exhibit enzymatic activity, which can be used as a label (for example, alkaline phosphatase, β-galactosidase, and the like), or can have rely solely on binding (for example, as an epitope or ligand-binding partner), or can include both enzymatic and ligand-binding features.

The presence of a surface marker permits one to quickly separate host cells that express the test gene (and thus the surface marker) from those that do not. Such separation can be effected by means of FACS (fluorescence-activated cell sorting), affinity panning, affinity column separation, and the like. Thus, one can identify host cells that express the test gene without the need to identify another phenotype or altered characteristic that results from the test gene expression. Further, one can separate host cells in which expression is regulated from cells in which expression is either constitutive or non-existent, by selecting cells that do not express the surface marker when the promoter is repressed or not induced, and from that pool selecting cells that express the marker following induction of the promoter. These cells can also be removed by using an antibody specific for the surface marker in combination with complement. It is also possible to perform the selection steps in reverse order, or to repeat the steps several times, although one may need to wait a sufficient period of time for marker present on the host cell surface to be cleared. Additionally, one can select several different pools of cells by using different methods for inducing the promoters, for example, where the vector is cloned into position adjacent to a plurality of different promoters, or next to promoters randomly. For example, one can select a pool of cells that do not express the surface marker constitutively, and from this pool select a subset of cells that express the surface marker in response to a change in temperature. The cells that were not selected can be subjected to other conditions, for example the presence or absence of a nutrient, and any cells that respond to such conditions are then selected.

The table below illustrates the advantages of the method of the invention over conventional procedures.

| | Conventional | pFastFind |
|---|---|---|
| Cell Cloning | Technology | Method |
| Time Elapsed | 35 days | 30 days |
| Manpower | 14 days | 5 days |
| Screening method on clonal cell lines | RT-PCR | FACS |
| Number of clones analyzed | 21 | 23 |
| Number of regulated clones | 1 | 10 |
| Cloning efficiency | 4.8% | 43.5% |

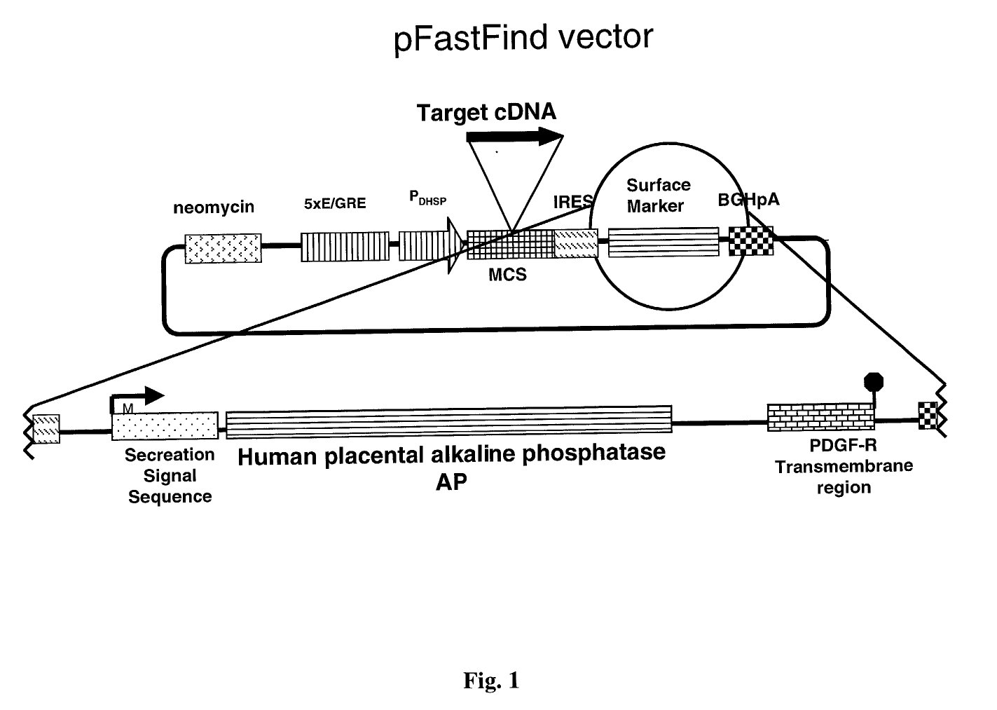

FIG. 1 is a schematic view of an embodiment of the invention (the pFastFind vector, SEQ ID NO:5). It highlights the neomycin resistance gene (herring-bone), the ecdysone inducible promoter (vertical hatching), the multiple cloning site (“MCS”) (grid), internal ribosome initiation sequence (IRES) (diagonal hatching), polyadenylation sequence (check hatching), secretion signal sequence (stippled hatching), surface marker alkaline phosphatase (horizontal hatching), and the transmembrane region from the PDEF receptor (brick pattern). The “M” indicates a methionine start site for translation of the surface marker, and the stop sign indicates the stop sequence for translation of the surface marker. the target cDNA or query gene is inserted in the MCS.

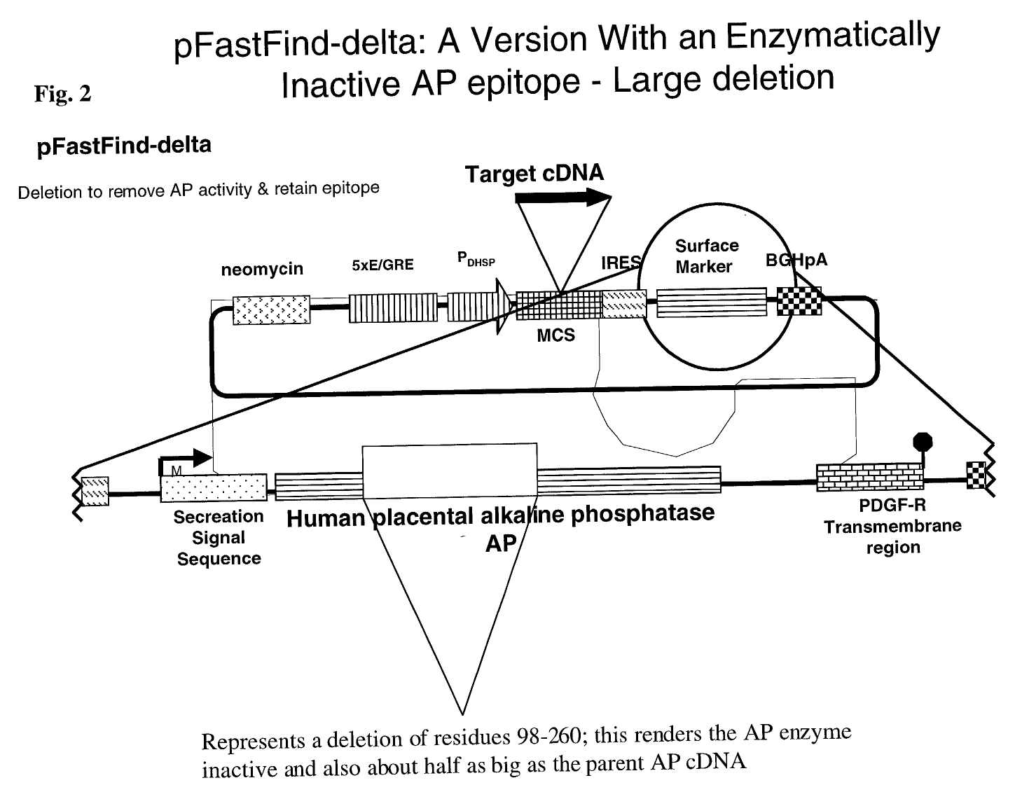

FIG. 2 is a schematic view of another embodiment of the invention (the pFastFind-delta vector, SEQ ID NO:6). Its highlighted features are depicted as in FIG. 1. The deletion of residues 98-260 of the surface marker is shown as a white box.

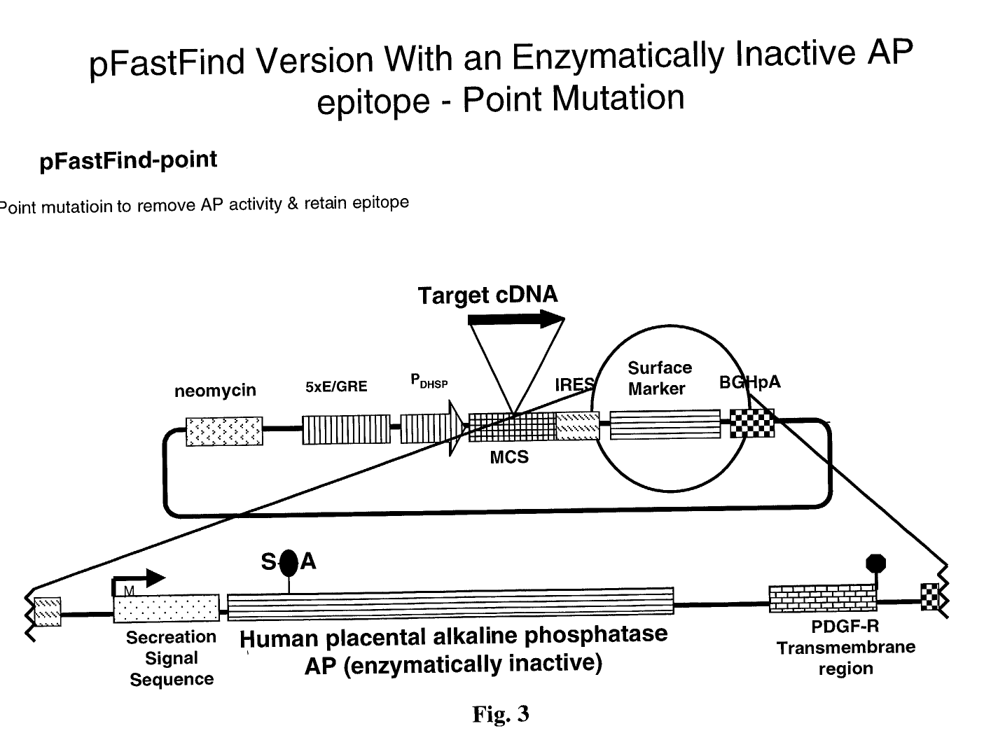

FIG. 3 is a schematic view of another embodiment of the invention (the pFastFind-point vector, SEQ ID NO:7). Its highlighted features are depicted as in FIG. 1. S→A indicates the point mutation that eliminates the catalytic activity of the alkaline phosphatase surface marker.

### EXAMPLES

The following examples are provided as a guide for the practitioner of ordinary skill in the art. Nothing in the examples is intended to limit the claimed invention. Unless otherwise specified, all reagents are used in accordance with the manufacturer's recommendations, and all reactions are performed at standard temperature and pressure.

### Example 1

### pFastFind-JNK3

(A) Construction of Vector pFastFind-JNK3

1. Building pIND-IRES: The following oligonucleotides were used to PCR amplify the IRES sequence from pIRES-EYFP (Clontech):

5′TCATCATCATCTCGAGCCAATTCCGCCCCTCTCCCTC (SEQ ID NO:1);

5′TCTCTCTAGACCGGGTTGTGGCAAGCTTATCATC (SEQ ID NO:2).

The resulting IRES cDNA was digested with XhoI and XbaI and ligated to pIND (Invitrogen) treated with XhoI and XbaI and calf intestine phosphatase (CIP). The ligated plasmid was transformed and pIND-IRES was isolated and verified by restriction digests.

2. Building pIND-IRES-SEAP: Full length secreted alkaline phosphatase (SEAP) cDNA was amplified from pSEAPBasic2 (Clontech) using primers containing NheI at the 5′ end and XbaI at the 3′ end. The resulting SEAP cDNA was digested with NheI and XbaI and ligated to pIND-IRES treated with XbaI and CIP. The ligated plasmid was transformed and pIND-IRES-SEAP was isolated and verified by restriction digests.

3. Building pIND-IRES/SEAP-TM (pFastFind): The following oligos were used to amplify the TM fragment from pDisplay (Invitrogen):

5′CTCTAGTCTAGAGTCGGGGCGGCCGGCCGCTTCGAGCAGACATCTCCCGGGAATCGCGGCTGCAG (SEQ ID NO:3);

5′TAGCTAGCTGATCTCGAGCGGCCGCCTGAACGT (SEQ ID NO:4).

The resulting TM PCR product was topo cloned into pcDNA3.1 and isolated by digestion with XbaI and NheI. The XbaI-TM-NheI fragment was ligated to pIND-IRES/SEAP treated with XbaI and CIP. The ligated plasmid was transformed and pIND-IRES/SEAP-TM was isolated and verified by restriction digests.

4. Building pFastFind-JNK3: pFastFind-JNK3 (SEQ ID NO:8) was constructed by inserting the protein coding sequence of JNK3 into pFastFind. The JNK3 sequence was isolated using polymerase chain reactions (Stratagene) from a human brain cDNA library using primers that encode a mammalian translation consensus sequence and the coding sequence surrounding the initiator methionine and stop codons of JNK3 (Genbank Accession number U07620). The PCR product was first subcloned into pIND, amplified by PCR, inserted into pcDNA2.1-TOPO and then subcloned in between SpeI and EcoRV restriction sites of pFastFind. The DNA sequence of the pFastFind-JNK3 vector was determined throughout the JNK3 coding region. The protein sequence of the JNK3 clone agreed with the consensus alignment of JNK3 clones present in Genbank with the exception of the following changes: Tyr to Cys at residue 240.

(B) Use of pFastFind-JNK3 to Isolate Clonal Cell Lines with Regulated Expression of JNK3

JNK3 is a member of the MAP kinase family of protein kinases. JNK3 shares sequence homology and common biochemical substrates with its close relative JNK1 (an important regulator of early response genes); however, its role in cell physiology is unclear. An understanding of JNK3 role in cell physiology is of immediate interest. To study this gene, we constructed a cell line in which JNK3's expression and activity could be regulated by the application of an exogenous agent that is inert to the engineered cell line.

Cell lines were isolated using either of two protocols. Protocol A (48-58 days) comprised: (a) transfecting cells with pFastFind; (b) selecting the transformed cells with neomycin for 15 days; (c) adding ponasterone A for 1 day; (d) isolating single cells displaying the surface marker by FACS; (e) allowing the single cells to multiply for 30-40 days; and (f) analyzing single clones treated with and without ponasterone A by FACS. Protocol B (59-79 days) comprised: (a) transfecting cells with pFastFind; (b) selecting the transformed cells with neomycin for 15 days; (c) adding ponasterone A for 1 day; (d) isolating an enriched pool of single cells displaying the surface marker by FACS; (e) allowing the single cells to multiply for 10-20 days; (f) adding ponasterone A for 1 day; (g) isolating single surface-marker positive cells using FACS; (h) allowing the single cells to multiply for 30-40 days; and (i) analyzing single clones treated with and without ponasterone A by FACS.

The pFastFind-JNK3 vector was transfected into ECR-293 cells (Invitrogen), and treated with neomycin for 15 days. Surviving cells were treated with 5 μM ponasterone A and rendered into single cell suspension by trypsinization. The cell suspension was incubated with anti-SEAP monoclonal antibody for 1 hour at room temperature and subsequently incubated with fluorescein-conjugated anti-mouse IgG for 30 minutes at room temperature in dark. The labeled cell suspension was resuspended into phosphate buffered saline containing 5 μg/ml of propidium iodide and analyzed for fluorescein fluorescence using CellQuest software on FACSVantageSE (Becton Dickinson). Cells with fluorescein fluorescence>20-30 and propidium iodide fluorescence<100 were gated and collected as population or single cell clones using CloneCyt software.

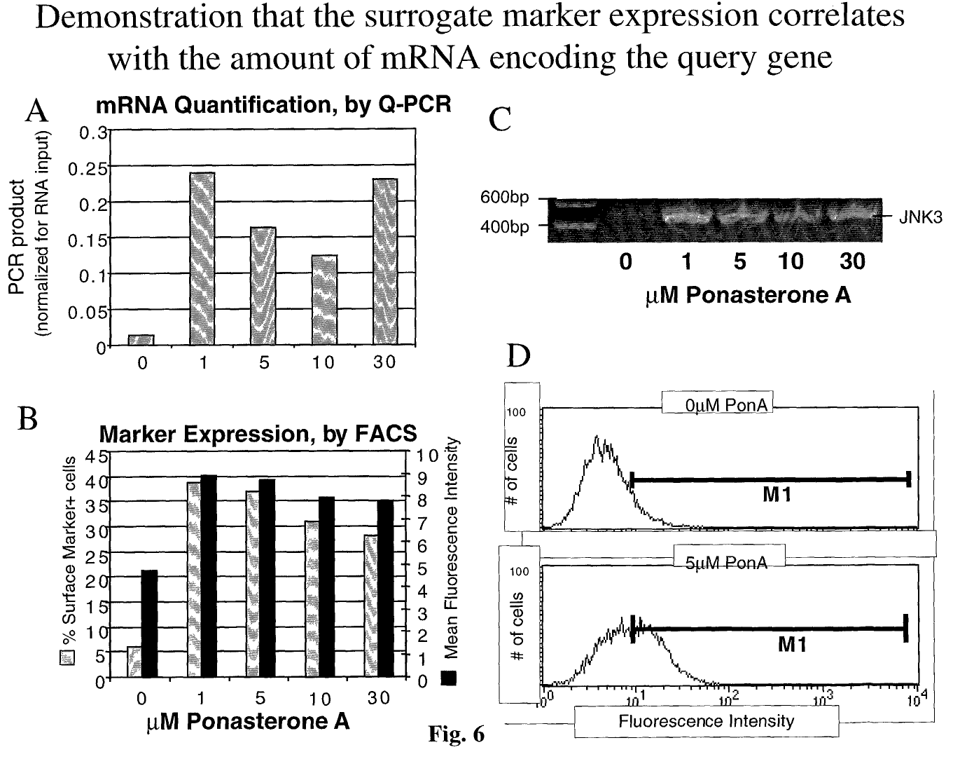

Single cells were expanded and then re-analyzed using FACS for their expression of the surface marker after treatment with ponasterone A for 24-48 h. Cells showing increased expression of the surface marker in the presence of ponasterone A as compared to unstained controls were analyzed at varying times (0-48 h) after the start of ponasterone A treatment and were subjected to increasing doses (0-30 μM) of ponasterone A. FIG. 6 shows one clone as an example where the expression of the surface marker is significantly increased with time after the addition of ponasterone A as well as with increasing concentrations of ponasterone A. Cell clones showing increased expression of the surfaces marker were also analyzed using semi-quantitative RT-PCR with primers specific for the query gene (JNK 3). FIG. 7 shows an example where the expression of the query gene increases in a similar fashion as the expression of the surface marker after the addition of ponasterone A. This shows that the methods described here can lead to a rapid selection of cell clones regulating the expression of the query gene.

What is claimed:

1. A polynucleotide, comprising:
a regulatable promoter;
a test gene;
an IRES sequence; and
a surface marker coding sequence, said surface marker comprising a secretion signal sequence, a detectable label protein, and a membrane anchor; wherein expression of said test gene also results in expression of said surface marker.

2. The polynucleotide of claim 1, wherein said surface marker does not exhibit enzymatic activity.

3. The polynucleotide of claim 1, further comprising a selectable marker.

4. The polynucleotide of claim 1, further comprising a heterologous origin of replication.

5. A host cell, comprising:
a polynucleotide, said polynucleotide comprising a regulatable promoter, a test gene, an IRES sequence, a surface marker coding sequence, said surface marker comprising a secretion signal sequence, a detectable label protein, and a membrane anchor; wherein expression of said test gene also results in expression of said surface marker.

6. The host cell of claim 5, wherein said test gene comprises a heterologous DNA sequence.

7. The host cell of claim 5, wherein said promoter is native to said host cell.

8. The host cell of claim 5, wherein said host cell is selected from the group consisting of yeast and mammalian cells.

9. A method of identifying a host cell that exhibits regulated expression of a test gene, comprising:
a) providing a plurality of host cells, each host cell comprising a polynucleotide, said polynucleotide comprising in order a regulatable promoter; a test gene; an IRES sequence; and a surface marker coding sequence, said surface marker comprising a secretion signal sequence, a detectable label protein, and a membrane anchor; wherein expression of said test gene also results in expression of said surface marker;
b) inducing said promoter; and
c) selecting a host cell that displays said surface marker on its surface.

10. The method of claim 9, further comprising:
d) repressing said promoter; and
e) selecting a host cell that no longer displays said surface marker on its surface.

11. The method of claim 9, wherein said host cell is selected by fluorescence-activated cell sorting.

12. A method of identifying a host cell that exhibits regulated expression of a test gene, comprising:
a) providing a plurality of host cells, each host cell comprising a polynucleotide, said polynucleotide comprising in order a regulatable promoter; a test gene; an IRES sequence; and a surface marker coding sequence, said surface marker comprising a secretion signal sequence, a detectable label protein, and a membrane anchor; wherein expression of said test gene also results in expression of said surface marker;
b) selecting a plurality of host cells that do not exhibit said surface marker in the absence of induction of said promoter;
c) inducing said promoter; and
d) selecting a host cell that exhibits expression of said surface marker.

13. The method of claim 12, wherein said host cell is selected by fluorescence-activated cell sorting.


SEQUENCE LISTING (8 sequences)

SEQ ID NO: 1 - DNA, 37 bp, PCR primer
tcatcatcatctcgagccaattccgcccctctccctc

SEQ ID NO: 2 - DNA, 34 bp, PCR primer
tctctctagaccgggttgtggcaagcttatcatc

SEQ ID NO: 3 - DNA, 65 bp, PCR primer
ctctagtctagagtcggggcggccggccgcttcgagcagacatctcccgggaatcgcggctgcag

SEQ ID NO: 4 - DNA, 33 bp, PCR primer
tagctagctgatctcgagcggccgcctgaacgt

SEQ ID NO: 5 - DNA, 7563 bp, pFastFind
agatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggagtaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatactttcaacaagttaccgagaaagaagaactcacacacagctagcgtttaaacttaagcttggtaccgagctcggatccactagtccagtgtggtggaattctgcagatatccagcacagtggcggccgctcgagccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccggtctagcccgggctcgagatctgcgatctaagtaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatctcccgggaatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgagatcagctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggcgcctatgtataaacttacataaatctttttatttgtttatccccaaggcgcgtgtaaaggggcttttcacggtggactgcagctgcctagccc

SEQ ID NO: 6 - DNA, 7469 bp, pFastFind-delta
agatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggagtaccctcgaccgccggagtataaatagaggcgcttcgtctacggagcgacaattcaattcaaacaagcaaagtgaacacgtcgctaagcgaaagctaagcaaataaacaagcgcaggacttgttcgatttgttagacgtcatttcacgttcaatttcacttagttaattttcattggtcgttggttcatttagttgacgttgatgactttagacggttcttcattaataacttatgttcttctcttgagacttatgaaagttgttgttaccgagaaagaagaactcacacacagctagcgtttaaacttaagcttggtaccgagctcggatccactagtccagtgtggtggaattctgcagatatccagcacagtggcggccgctcgagccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccggtctagcccgggctcgagatctgcgatctaagtaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacgctggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatctcccgggaatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgagatcagctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcggg

SEQ ID NO: 7 - DNA, 6980 bp, pFastFind-point
agatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggagtaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatactttcaacaagttaccgagaaagaagaactcacacacagctagcgtttaaacttaagcttggtaccgagctcggatccactagtccagtgtggtggaattctgcagatatccagcacagtggcggccgctcgagccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccggtctagcccgggctcgagatctgcgatctaagtaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcaggggcgggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatctcccgggaatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgagatcagctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcggg

SEQ ID NO: 8 - DNA, 8750 bp, pFastFind-JNK3
agatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggagtaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatactttcaacaagttaccgagaaagaagaactcacacacagctagcgtttaaacttaagcttggtaccgagctcggatccactagtacaaccatggtgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctgcaaggagaacgtggatatatggtctgtgggatgcattatgggagaaatggttcgccacaaaatcctctttccaggaagggactatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcagcacaggtgcagcagtgagactacaaggacgatgatgacgccgatatccagcacagtggcggccgctcgagccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccggtctagcccgggctcgagatctgcgatctaagtaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatctcccgggaatccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgagatcagctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcggg